(19) United States
(12) Patent Application Publication
(10) Pub. No.: US 2002/0111540 A1
(43) Pub. Date: Aug. 15, 2002

(54) Method, medical system and portable device for determining psychomotor capabilities

(76) Inventors: Volker Schmidt, Erlangen (DE); Werner Striebel, Schwarzenbruck (DE)

Correspondence Address: YOUNG & THOMPSON, 745 SOUTH 23RD STREET 2ND FLOOR, ARLINGTON, VA, 22202

(21) Appl. No.: 10/055,032

(22) Filed: Jan. 25, 2002

(30) Foreign Application Priority Data
Jan. 25, 2001 (DE) 10103326.5

(51) Int. Cl. A61B 5/00

(52) U.S. Cl. 600/300; 128/904; 434/236; 704/231

(57) ABSTRACT

A method and a medical system for determining psychomotor capabilities of a patient (1) in an extraclinical environment (5), having a data bank (3), which is arranged at a location (6) other than the location (5) at which a psychomotor test for determining the psychomotor capabilities is carried out, and which stores data on the patient (1) determined during the psychomotor test. The invention also relates to a portable device for determining psychomotor capabilities of a patient, having a writing surface (21) on which handwritten letters or numbers can be written, elements (22) for recognizing the handwritten letters or numbers, a viewing area (23) for reproducing the recognized handwritten letters or numbers as printed letters and elements (24) for connection to the Internet.

### DESCRIPTION

The invention relates to a method, a medical system and a portable device for determining psychomotor capabilities of a patient.

In the medicinal treatment of a patient with substances attacking the patient's nervous system, it is necessary to check psychic and motor capabilities of the patient during the treatment, in particular to detect under- or overdoses in time and to minimize side-effects of the substances. Substances attacking the central nervous system include sedatives (sleeping tablets), amphetamines (stimulants, appetite suppressants), neuroleptics, antidepressants and antiepileptics. The effects of these substances may include a noticeable change in vigilance, reaction time, the intricate and basic motor systems and the memory of the patient.

To ensure appropriate treatment of the patient, the concentration of the substances taken can be determined, for example, in the blood of the patient by laboratory techniques. In addition, special tests can be carried out in hospital or a doctor's surgery, for example to determine the patient's reaction time, psychomotor coordination capability and orientation capability in terms of time and place.

In particular in the case of repeated tests, the patient must frequently visit the hospital or the doctor's surgery, which takes up a greater amount of time for the patient. Therefore, some patients are inclined to have only a few of these tests carried out or to miss appointments with the doctor, which in turn has an adverse effect on the successes of treatment.

The invention is therefore based on the object of specifying a method and designing a medical system in such a way that it can be used to create preconditions for carrying out tests to determine psychomotor capabilities of the patient more practically for the patient.

According to the invention, this object is achieved by a method for determining psychomotor capabilities of a patient in an extraclinical environment, having the following method steps:

a) carrying out a psychomotor test and

b) transmitting the data obtained during the psychomotor test to a data bank which is arranged at a location other than the location at which the psychomotor test is carried out.

According to the invention, a test for determining psychomotor capabilities of the patient is consequently carried out in an extraclinical environment. An extraclinical environment is understood as meaning that the psychomotor test is not carried out in a doctor's surgery or in hospital, but for example at the patient's home, the patient's place of work or else outdoors. This dispenses with the time-consuming visits to the doctor's surgery or hospital for the patient. The data obtained during the test are subsequently transmitted to the central data bank, which is arranged for example at the surgery of a doctor treating the patient. On account of the central storage of the data, it is possible in particular for a number of other persons, in particular other doctors treating the patient, to have access to the data.

According to one embodiment, the data bank is operated by a service provider. Such a service provider can maintain the data bank or, in particular, provide further services in a particularly favorable way. For example, it is conceivable for the service provider to operate a call center, to which questions from the patient or the doctor can be directed. Consequently, patient care can be made more effective and, in particular, if there are questions, further help can be provided in a quick, friendly and reliable way.

In a further embodiment of the invention, it is provided that the psychomotor test is carried out using a telephone, a computer which can be connected to the Internet and/or a Personal Digital Assistant (PDA). A Personal Digital Assistant is understood as meaning a portable electronic device into which data can be entered, the Personal Digital Assistant having means for further processing and storing these data. Examples of a Personal Digital Assistant are a palmtop or portable computer (laptop). The patient can, for example, be contacted by telephone by a person working at the call center or automatically by the data bank, which is provided with suitable means for voice input and voice reproduction, or the patient can himself telephone a person at the call center or the data bank, in order that he carries out the psychomotor test over the phone. In particular if a telephone is used, the test can be carried out at virtually any desired locations, such as for example at the patient's home, in a telephone booth or else outdoors, in particular if the patient has a cellphone. Consequently, the patient is virtually unrestricted as to where the test is carried out. The patient therefore only has to take a negligible amount of time, for actually carrying out the test, as a result of which the patient is more inclined to carry out this test regularly, in particular, or generally more often. This consequently creates a precondition for better treatment of the patient.

According to a further variant of the invention, the psychomotor test comprises a reaction test, a memory test, a test for determining the legibility of the handwriting of the patient and/or a test for determining the intelligibility of the voice of the patient. In order for example to test the reaction time of the patient, he can be given numbers over the telephone, whereupon the patient presses as quickly as possible the button or buttons on the telephone corresponding to the number. The patient's memory can be similarly tested, in that for example the patient is given a sequence of numbers and the patient then presses the corresponding buttons on the telephone. The intelligibility of the patient's voice can be tested, for example, by the patient repeating a sentence or sequence of words given to him over the phone. Similar tests can also be carried out with a computer which can be connected to the Internet. By means of the Personal Digital Assistant, it is possible, for example, for data obtained during the psychomotor test to be recorded by the patient or a person looking after the patient and transmitted to the data bank later, for example via the Internet. With the Personal Digital Assistant, a test for checking the legibility of the patient's handwriting can be carried out if the Personal Digital Assistant has a writing surface on which the patient writes individual letters by hand, for example with a pen, and the Personal Digital Assistant is configured in such a way that it recognizes the handwritten letters and reproduces them as printed letters on a viewing area. If the psychomotor system is dysfunctional, it is possible that the patient's handwriting is illegible and the Personal Digital Assistant recognizes only a few handwritten letters. Consequently, an error rate which can be calculated from the number of unrecognized letters in comparison with the number of letters entered is a measure of the psychomotor capability of the patient.

According to a variant of the invention, the data are evaluated, in particular automatically, by an evaluation device assigned to the data bank. The evaluation device may, according to an advantageous variant of the invention, in this case have means for voice pattern analysis, means for time measurement, means for comparing sequences of letters, means for comparing sequences of numbers and/or an expert system. With the means for voice pattern analysis, the test for the intelligibility of the patient's voice can be carried out in particular, in that the patient repeats at least one sentence or sequence of words spoken to him by the data bank and the intelligibility of the repeated sentence or repeated sentences or sequence of words is or are automatically evaluated. With the means for time measurement, which may for example comprise a stopwatch, the reaction time of the patient can be determined. The sequences of letters or sequences of words can be evaluated by the means for comparing sequences of letters or sequences of numbers.

According to a preferred embodiment, it is possible to contact the data bank via the Internet. Consequently, it is possible for example to interrogate the data bank, and consequently ask for the data stored in the data bank, via the Internet, whereby in particular the doctors treating the patient have access to important data on the patient quickly and in a simple way.

According to a further variant of the invention, the patient and/or at least one other person is informed, in particular automatically, if the data evaluated give cause for concern. The other person may be, for example, the doctor treating the patient, who, on the basis of the message, can check the data and, if necessary, quickly and reliably initiate appropriate measures for treating the patient.

A further embodiment of the invention provides that the data bank automatically informs the patient and/or at least one other person if, after a psychomotor test, a predetermined time period has elapsed without a further psychomotor test having been carried out. This creates a precondition allowing the patient to be reminded of the importance of the psychomotor test, in particular if he inadvertently forgot to carry out the psychomotor test.

The object is also achieved by a medical system for determining psychomotor capabilities of a patient in an extraclinical environment, having a data bank, which is arranged at a location other than the location at which a psychomotor test for determining the psychomotor capabilities is carried out, and which stores data on the patient determined during the psychomotor test, an evaluation device assigned to the data bank for evaluating these data and an alarm device for generating an alarm signal if the data evaluated give cause for concern. The medical system is consequently characterized in that a psychomotor test can be carried out in particular at the home, at the place of work or else outdoors instead of at a doctor's surgery or in hospital. The data obtained during the psychomotor test are subsequently transmitted to the central data bank. The data bank may be arranged, for example, at the surgery of the doctor treating the patient or a hospital. Consequently, the patient need not visit a doctor's surgery or hospital in order to carry out the psychomotor test, but can carry it out conveniently, for example at home. Transmitting the data obtained during the test to the central data bank has the effect in particular of giving the doctor treating the patient the possibility of viewing the data obtained during the test and modifying the treatment of the patient accordingly.

Further advantageous refinements of the medical system emerge from the subclaims.

The object is also achieved by a portable device for determining psychomotor capabilities of a patient in an extraclinical environment, having a writing surface on which handwritten letters or numbers can be written, means for recognizing the handwritten letters or numbers, a viewing area for reproducing the recognized handwritten letters or numbers as printed letters and means for connection to the Internet, the psychomotor capabilities being determined by a test for checking the legibility of the handwriting of the patient. The patient writes numbers, or preferably letters, by hand, for example with a pen, on the writing surface of the portable device. If the handwriting is legible, the numbers or letters are recognized by the means for recognizing the handwritten letters or numbers and reproduced on the viewing area as printed letters. The patient or a person looking after the patient notes the number of letters or numbers written and the number of letters or numbers not recognized and calculates from this an error rate. In particular, the error rate can be transmitted by the portable device, if it can be connected to the Internet, to a central data bank for example.

According to an advantageous embodiment, the portable device is configured in such a way that it automatically calculates the error rate.

An exemplary embodiment of the invention is represented in the attached schematic drawings, in which:

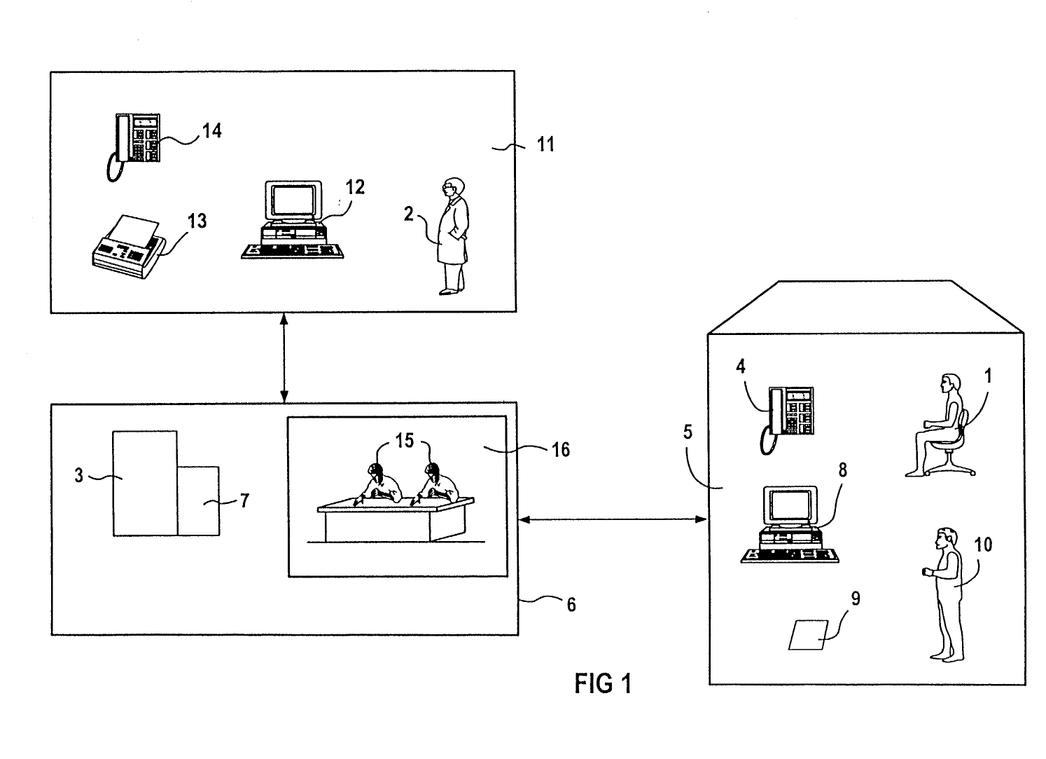

FIG. 1 shows a diagram comprising the medical system according to the invention and

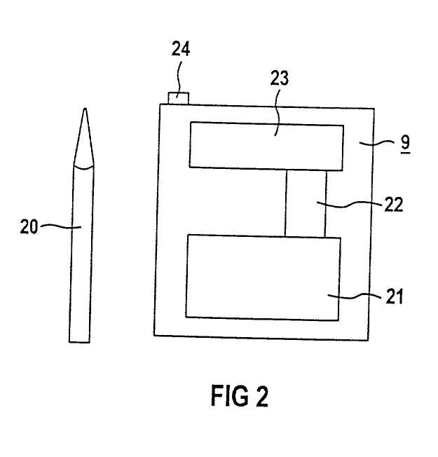

FIG. 2 shows a Personal Digital Assistant (PDA).

A patient 1, schematically represented in FIG. 1, regularly takes a medicine, not represented in FIG. 1, attacking his central nervous system. In order that a doctor 2 treating the patient 1 can detect an under- or overdose of the medicine or side-effects of the medicine as quickly as possible, the patient 1 carries out a psychomotor test, in which, in the case of the present exemplary embodiment, the reaction time, the memory, the intelligibility of the voice and the legibility of the handwriting of the patient 1 is tested, regularly, in the case of the present exemplary embodiment three times a week, in an extraclinical environment. In particular in the case of side-effects or an overdose of the medicine, it is to be expected that, inter alia, the reaction time, the memory, the intelligibility of the voice and/or the legibility of the handwriting of the patient 1 will deteriorate.

In order that the psychomotor test can be carried out in a way which is practical for the patient 1, when he wishes to carry out the psychomotor test the patient 1 uses a telephone 4 to contact a data bank 3, which in the case of the present exemplary embodiment is provided with means suitable for voice input and voice reproduction. In the case of the present exemplary embodiment, the patient 1 is at his home 5 during the psychomotor test. It goes without saying that the patient 1 can also carry out this test, for example, at the workplace, outdoors, such as for example with a cellphone not represented in FIG. 1, or else in a telephone booth, as long as he has access to a telephone.

In the case of the present exemplary embodiment, the data bank 3 is operated by a service provider 6, which maintains the data bank 3. Furthermore, the data bank 3 is assigned an evaluation device 7, which evaluates the data obtained on the basis of the psychomotor test.

When the data bank 3 is contacted, firstly the identity of the patient 1 is determined by means of an identification number combination, which the patient 1 gives over the telephone 4 and the data bank 3 evaluates with a suitable program procedure. Subsequently, in the case of the present exemplary embodiment, a reaction test is carried out, in that the data bank 3 starts a test program stored in the data bank 3 and, by means of the telephone 4, gives the patient 1 a number between 0 and 9, which the patient 1 confirms with the dialing buttons of the telephone 4. This test is repeated nine times with different numbers. For the reaction test, the evaluation device 7 measures the respective reaction time which the patient 1 requires to press the corresponding dialing button of the telephone 4 after the number is given, and compares this reaction time with an average reaction time which the patient 1 required for the same test before the treatment with the medicine, stored in the data bank 3. Should the reaction times in the case of the present embodiment have deteriorated by more than one second in three measurements, the result of the reaction test is classified by the evaluation device 7 as giving cause for concern, and otherwise as giving no cause for concern. What is more, the individual reaction times, the changes in the reaction times in comparison with the average reaction time determined before the treatment and the result of the reaction test are stored in the data bank 3.

Subsequently, in the case of the present exemplary embodiment, a test for determining the intelligibility of the voice of the patient 1 is carried out, in that the data bank 3 starts a further test program stored in the data bank 3 and speaks to the patient 1 a sentence stored on the data bank 3, which the patient 1 repeats. The sentence repeated by the patient 1 is recorded by the data bank 3 and checked by the evaluation device 7 by means of a voice pattern analysis for completeness and intelligibility and is compared with a voice pattern analysis of the patient 1 which was carried out before the treatment with the medicine. When there is a clear deterioration in the intelligibility of the voice or the sentence is incomplete, the test for voice intelligibility is classified as giving cause for concern, and otherwise as giving no cause for concern. The result of this test is likewise stored in the data bank 3.

After that, in the case of the present exemplary embodiment, a memory test is carried out, in that the data bank 3 starts a further test program, stored in the data bank 3, and gives the patient 1 a ten-digit number, which the patient reproduces by means of the dialing buttons. The evaluation device 7 in this case compares the ten-digit number given by the database 3 with the ten-digit number entered by the patient 1 and determines the number of wrongly entered digits. Subsequently, the evaluation device 7 compares the number of wrongly entered digits with an average number of wrongly entered digits for a series of the same tests which the patient 1 carried out before the treatment with the medicine. Should the number of wrongly entered digits have increased by more than two, in the case of the present exemplary embodiment the memory test is classified by the evaluation device 7 as giving cause for concern, and otherwise as giving no cause for concern. The number of wrongly entered digits and the result of the memory test are stored in the data bank 3.

Another possible way of carrying out a psychomotor test in an extraclinical environment is to use a computer 8 connected to the Internet. In the case of the present exemplary embodiment, the patient 1 uses the computer arranged at his home 5 and connected to the Internet to contact a WWW home page assigned to him and maintained by the service provider 6 and carries out a memory test. In the case of the present exemplary embodiment, after contacting the WWW home page, the patient 1 has a time of one minute to read a sentence reproduced on the WWW home page. After one minute, this sentence is masked out and the patient 1 must enter the sentence into the computer 8 by means of a keyboard assigned to the computer 8, and the sentence is also stored in the data bank 3. Subsequently, the evaluation device 7 compares the sentence entered by the patient 1 with the original sentence and determines the error rate, which in turn is compared with an error rate of the patient 1 which was determined before the treatment with the medicine. If, in the case of the present exemplary embodiment, the error rate increases by more than 10%, the memory test is classified as giving cause for concern, and otherwise as giving no cause for concern.

A third form in which a psychomotor test is carried out in an extraclinical environment is a test for determining the legibility of the handwriting of the patient 1, which is carried out by means of a Personal Digital Assistant (PDA) 9, likewise schematically represented in FIG. 2. In the case of the present exemplary embodiment, the PDA 9 is made available to the patient 1 by a person 10 looking after the patient 1. By means of a pen 20, schematically represented in FIG. 2, the patient 1 writes by hand on a writing surface 21 of the PDA 9 various letters given by the person 10, which are recognized by suitable means 22 for recognizing handwritten letters of the PDA 9 when there is an only slightly dysfunctional psychomotor capability of the patient 1 and reproduced as printed letters on the viewing area 23 of the PDA 9. The person 10 notes during this test the number of illegibly written letters, the number of letters given and the time period which the patient 1 requires for the writing of the letters. After completion of the test for determining the legibility of the handwriting of the patient 1, the person 10 in the case of the present exemplary embodiment transmits the results of the writing test to the data bank 3 via the Internet. For this purpose, the PDA 9 is provided with a suitable interface 24 for the connection to the Internet. Subsequently, the evaluation device 7 calculates from this information an error rate, which the evaluation device 7 compares with an error rate of the patient 1 determined before the treatment with the medicine. What is more, the evaluation device 7 compares the time period which the patient 1 required for writing the letters with a time period which the patient 1 required for writing the letters without medicinal treatment. In the case of the present exemplary embodiment, the evaluation device 7 classifies the result of the test for the legibility of the handwriting of the patient 1 as giving cause for concern if the error rate and/or the time period for writing the letters increase by more than 10%. Otherwise, the evaluation device 7 classifies the writing test as giving no cause for concern.

As an alternative to the manual writing of the number of illegible letters, the number of letters given and the time period required for writing the letters, the PDA 9 may also be configured in such a way that it automatically acquires this information or automatically calculates the error rate.

Should one of the tests described above have been classified as giving cause for concern by the evaluation device 7, the data bank 3 automatically sends an e-mail to a computer 12, arranged in the case of the present exemplary embodiment at a surgery 11 of the doctor 2, in order to inform the doctor 2 of the result giving cause for concern. In particular in the event of a disrupted Internet connection, the doctor 2 can also be informed of the psychomotor test giving cause for concern by one of the persons 15 working at a call center 16 assigned to the service provider 6 by means of a fax machine 13 or telephone 14 arranged at the doctor's surgery 11. The persons 15 can also be contacted, inter alia, if the doctor 2, the patient 1 or the person 10 has questions concerning the psychomotor test.

What is more, it is provided in the case of the present exemplary embodiment that the doctor 2 can contact the data bank 3 via the Internet in order for example to check the psychomotor test, in particular on account of a result giving cause for concern, and if necessary contact the patient 1 and/or quickly and reliably initiate remedial measures for the patient 1.

Furthermore, it is provided in the case of the present exemplary embodiment that the data bank 3 automatically informs the doctor 2 by e-mail, by telephone or by fax if more than two days have elapsed after a psychomotor test without the patient 1 having carried out a new psychomotor test. Consequently, in such a case the doctor 2 can remind the patient 1 of the importance of the psychomotor test, in order to ensure good treatment of the patient 1 with the medicine.

Moreover, the service provider 6 does not necessarily have to operate a call center 16. The data bank 3 also does not necessarily have to be operated by a service provider 6, it may for example also be arranged at the doctor's surgery 11 and be looked after by the doctor 2 or a customer service provider.

It is also possible for one of the persons 15 at the call center 16 or the data bank 3 to contact the patient 1, in order that the patient carries out the psychomotor test.

The evaluation device 7 may also comprise an expert system, which interprets the data transmitted on account of the psychomotor test on the basis of control systems or on the basis of probabilities. In this case, an individualization of the expert system to the patient can be envisaged, i.e. the expert system gets to know the patient it is keeping under surveillance better and better during the surveillance process; for this purpose, as a learning system, it continually makes forecasts on expected future data, which it compares with the true data. In this way, an individualized surveillance is achieved.

The psychomotor test also does not necessarily have to be carried out regularly.

The description of the psychomotor test is to be understood as given by way of example. In particular, not all the devices mentioned, the telephone 4, computer 8 or PDA 9, have to be used for the psychomotor test. Other devices, not shown in FIGS. 1 and 2, or methods may also be used for carrying out the psychomotor test. It is also not necessary for the invention for the patient 1 to be looked after by the person 10. The configuration of a PDA 9 shown in FIG. 2 is also to be understood as given only by way of example. A PDA may, in particular, also be a laptop.

An automatic evaluation of the psychomotor test by means of the evaluation device 7 or automatic informing of the doctor 2 in the case of a result of the psychomotor test giving cause for concern is optional for the inventive method. Other persons may also be informed. Informing of the doctor that the patient 1 is not carrying out a psychomotor test is also optional both for the method and for the medical system. Furthermore, other persons may also be informed.

1. A method for determining psychomotor capabilities of a patient in an extraclinical environment (5), having the following method steps:
a) carrying out a psychomotor test and
b) transmitting the data obtained during the psychomotor test to a data bank (3) which is arranged at a location (6) other than the location (5) at which the psychomotor test is carried out.

2. The method for determining psychomotor capabilities as claimed in claim 1, in which the data bank (3) is operated by a service provider (6).

3. The method for determining psychomotor capabilities as claimed in claim 1 or 2, in which the psychomotor test is carried out using a telephone (4), a computer (8) which can be connected to the Internet and/or a Personal Digital Assistant (PDA) (9).

4. The method as claimed in one of claims 1 to 3, in which the psychomotor test comprises a reaction test, a memory test, a test for determining the legibility of the handwriting of the patient and/or a test for determining the intelligibility of the voice of the patient (1).

5. The method for determining psychomotor capabilities as claimed in one of claims 1 to 4, in which the data are evaluated, in particular automatically, by an evaluation device (7) assigned to the data bank (3).

6. The method for determining psychomotor capabilities as claimed in claim 5, in which the evaluation device (7) comprises means for voice pattern analysis, means for time measurement, means for comparing sequences of letters, means for comparing sequences of numbers and/or an expert system.

7. The method for determining psychomotor capabilities as claimed in one of claims 1 to 6, in which the data bank (3) can be contacted via the Internet.

8. The method for determining psychomotor capabilities as claimed in one of claims 5 to 7, in which the data bank (3) automatically informs the patient (1) and/or at least one other person (2) if the data evaluated give cause for concern.

9. The method for determining psychomotor capabilities as claimed in one of claims 1 to 8, in which the data bank (3) automatically informs the patient (1) and/or at least one other person (2) if, after a psychomotor test, a predetermined time period has elapsed without a further psychomotor test having been carried out.

10. A medical system for determining psychomotor capabilities of a patient (1) in an extraclinical environment (5), having a data bank (3), which is arranged at a location (6) other than the location (5) at which a psychomotor test for determining the psychomotor capabilities is carried out, and which stores data on the patient (1) determined during the psychomotor test, an evaluation device (7) assigned to the data bank (3) for evaluating these data and an alarm device (12 to 14) for generating an alarm signal if the data evaluated give cause for concern.

11. The medical system for determining psychomotor capabilities as claimed in claim 10, in which the evaluation device (7) has means for voice pattern analysis, means for time measurement, means for comparing sequences of letters, means for comparing sequences of numbers and/or an expert system.

12. The medical system for determining psychomotor capabilities as claimed in claim 10 or 11, in which the data bank (3) can be contacted via the Internet.

13. A portable device for determining psychomotor capabilities of a patient, having a writing surface (21) on which handwritten letters or numbers can be written, means (22) for recognizing the handwritten letters or numbers, a viewing area (23) for reproducing the recognized handwritten letters or numbers as printed letters and means (24) for connection to the Internet, the psychomotor capabilities being determined by a test for checking the legibility of the handwriting of the patient (1).

14. The portable device for determining psychomotor capabilities of a patient as claimed in claim 13, which is configured in such a way that it automatically calculates an error rate from the number of handwritten letters or numbers and the letters or numbers not recognized.
